(19) United States
(12) Patent Application Publication
(10) Pub. No.: US 2017/0003262 A1
(43) Pub. Date: Jan. 5, 2017

(54) APPARATUS AND METHOD OF ESTABLISHMENT OF THE PRESENCE AND MEASUREMENT OF THE CONTENT OF COINAGE METALS SUCH AS GOLD, SILVER, COPPER AND PLATINUM GROUP METALS AS WELL AS OTHER CHEMICAL ELEMENTS IN INDUSTRIAL ALLOYS, COATINGS AND DIELECTRIC COMPOUNDS

(71) Applicant: Nikita Balashov Katz, (US)

(72) Inventor: Nikita Balashov Katz, Salt Lake City, UT (US)

(21) Appl. No.: 15/199,994

(22) Filed: Jul. 1, 2016

Related U.S. Application Data
(60) Provisional application No. 62/187,662, filed on Jul. 1, 2015.

(51) Int. Cl. G01N 33/20; G01N 21/76; G01N 21/65

(52) U.S. Cl. CPC G01N 33/20; G01N 21/658; G01N 21/76

(57) ABSTRACT

An apparatus and a method of detection of the presence of coinage metals such as gold, silver and copper, as well as platinum group metals such as ruthenium, rhodium, palladium, osmium, iridium and platinum and the specific composition of alloys of aforementioned coinage and platinum group metals, especially alloys used in industry and jewelry production, both as macroscopic objects and in coatings are disclosed. The disclosed method is based on the use of surface enhanced physical and chemical phenomena that lead to the development of significant and measurable change in specific characteristics of a chemical compound when it is applied onto the surface of a metal alloy of the said coinage and platinum group metals. Effectively, the disclosed method may be described as the “inversion” of the traditionally used modality in which an unknown compound is applied onto the surface of a metal (or alloy of metals) of known composition, usually, chemically pure metal. In this inverted modality, the disclosed method is also applicable to testing of certain dielectric compounds.

One example of the disclosed method is the use of surface enhanced Raman spectroscopy (SERS) in which a solution, film or particles of a chemical compound with known SERS signature is applied on the surface of the material to be analyzed and a SERS spectrum is captured and processed with the use of a computing device. The resulting Raman spectrum is compared with the Raman spectrum of the compound in its bulk or unapplied form and presents with a specific degree of enhancement as well as changes in relative magnitude and position of the characteristic peaks due to surface interactions between the components of the analyte (gold, silver, copper, platinum group metals and other elements) with the functional groups of the applied chemical compound. Another aspect of the disclosed method is the set of characteristics that are utilized in choosing the reagent to be used in the SERS setup: the reagent compound is chosen to assure that enhancement and shifting of the peaks of the Raman spectrum depends on the presence and relative amounts of gold, silver, copper and platinum group metals in the alloy, and the intensities and relative position of the peaks correspond to the specific amounts of said metals in the alloy. Additionally, supplemental methods of enhancement of signal may be employed such as electrochemical enhancement of surface phenomena, resonance Raman and other plasmonic and quantum mechanical effects aiming to improve both strength of the signal and its specificity. Additionally, transfer of energy to the surface of the analyzed alloy as to increase the intensity of the signal may be employed, such as heating or irradiation with photons of different wavelength. Additionally, chemical processes that are affected by surface enhancement phenomena, such as chemiluminescence may be employed instead of surface enhanced Raman spectroscopy.

The disclosed apparatus is an exemplary embodiment of the disclosed method utilizing one of the varieties of the disclosed method, namely, surface enhanced Raman spectroscopy.

### REFERENCE TO RELATED APPLICATIONS

This application is related to U.S. Provisional application No. 62/187,662 filed on Jul. 1, 2015 to which priority is claimed under 35 USC 119.

### TECHNICAL FIELD

The present invention relates generally to techniques that establish the presence and measure the content of chemical elements especially inclusive of the coinage metals, namely gold (Au), silver (Ag) and copper (Cu), as well as the platinum group metals (PGM), inclusive of ruthenium (Ru), rhodium (Rh), palladium (Pd), osmium (Os), iridium (Ir), and platinum (Pt), in materials including samples of purified metals and elements, metal alloys and dielectric compounds both in the solid, macroscopic form and in the form of coatings, often of minute or microscopic thickness.

Such alloys and coatings are commonly used in industry, production of electronic components and devices, as well as jewelry fabrication.

Other applications of the invention are possible, as the invention offers a universal method of identification of chemical elements and measurement of their relative content in an alloy or dielectric material or an object fabricated from an alloy as well as a dielectric compound.

### BACKGROUND

The disclosed method and apparatus utilize the quantum-mechanical and chemical phenomenon known as surface enhancement, in which certain physical and chemical processes are notably enhanced if they take place on the surface of metallic elements and their alloys or the interface between certain types of dielectric materials and another material, such as a gas, a liquid or a solid. Additionally, the method and apparatus utilize the phenomenon of modification of the geometric shape, dimensions and characteristic vibrations of molecules that are coordinated or bound to a surface of a solid (inclusive of solids fabricated from pure metals and their alloys) or to the surface of a coating inclusive of coatings fabricated from metals and their alloys.

The principle of surface enhancement had been extensively used in the analytical chemistry setup that utilizes surfaces made from pure metallic elements, most commonly, the coinage metals such as gold, silver, and copper, and, in some cases, platinum group metals, onto which a tested substance is applied and subjected to physical and chemical processes which, due to surface enhancement phenomenon, present with different results from what is observed when the same processes take place without the surface enhancement, for example when the substance is present in the bulk or free form.

Said physical and chemical processes commonly include but are not limited to inelastic scattering of light, known as Raman scattering, an example of a physical process; as well as emission of light as a consequence of an energy-producing chemical reaction, known as chemiluminescence, an example of a chemical process.

Hundreds of US and international Letters Patent had been issued for the variations of such traditional and well-researched setup, with the most commonly utilized physical process being surface enhanced Raman scattering (also termed surface enhanced Raman spectroscopy, with the two denotations commonly used interchangeably) that is used to characterize the tested substance applied onto a surface of a metal or alloy of metals of known composition which may allow identification of various chemical groups present in the tested substance.

To date no inventor had claimed the use of the same principle for the purpose of characterization of the composition of the metallic surface itself by application of a substance with desirable characteristics that undergo a specific change due to surface enhancement. The aforementioned desirable characteristics include the presence of specific chemical groups, radicals and moieties, which, on the atomic level, are separated by distances that relate to the size of the lattice of the underlying surface.

This approach is particularly fruitful for the detection of presence and measurement of composition of alloys of coinage metals such as gold, silver and copper in various industrial and jewelry alloys, a task that is currently accomplished either in a destructive fashion, or with the use of potentially dangerous energetic electromagnetic radiation such as X-rays or gamma rays, or corrosive and toxic substances, or with significant subjective factor and corresponding subjective error.

The principle of the invention is in the use of the disclosed modality whereby: a Reagent (a solid, a liquid or a gas) with known and desirable properties in regard of surface enhancement is applied on the surface of a tested solid object; a physical or chemical process that is amendable to surface enhancement is initiated and observed with acquisition of relevant experimental data; the resulting data are compared with control data obtained from the sample of the same Reagent whilst not applied on a tested surface; the complete datasets from the experimental measurement according to paragraph 2 and the control measurement according to paragraph 3 are analyzed to establish the presence and relative amounts of constituents in the tested solid object.

The principle of the invention is hereby explicated using inelastic scattering of light commonly known as Raman scattering as an example of the relevant physical process.

The phenomenon of surface enhancement in Raman scattering refers to the dramatic increase in the number of photons scattered inelastically by a chemical compound that is applied to the surface of a metal. Without the surface enhancement, the proportion of inelastic (Raman) scattered light to the elastic (Rayleigh) scattered light is very small and detection of the inelastically scattered photons poses a significant technical problem.

Surface enhancement of Raman scattering was first observed by M. Fleischmann and co-authors in the 1970s (Citation 1). The initial discovery was that of great increase of the counts of inelastically scattered photons when a chemical substance (namely, pyridine) was applied onto a roughened surface of an electrode made from pure silver. Subsequent research has identified coinage metals (gold, silver, and copper, all belonging to Group 11 (1B) of the Periodic table) as well as platinum group metals (palladium and platinum, potentially ruthenium, rhodium, osmium, and iridium, all belonging to groups 8, 9, 10 (VIII) of the Periodic table) as particularly advantageous in regard of ability to generate the surface enhancement phenomenon when chemical compounds were applied onto surfaces of such metals and illuminated with monochromatic visible or ultraviolet light, for example, produced by a laser source (Citation 2). Certain dielectric compounds, described as truncated periodic multilayers or alternatively described as supporting Bloch surface waves, are also capable of generation of the surface enhancement phenomenon (Citation 3) and are, therefore, included in the overall scope of the disclosed invention.

Practically all scientific experiments and inventions in the field of surface enhanced Raman scattering had been concerned with the use of this phenomenon to characterize various chemical compounds and their reactions while these compounds were applied on surfaces prepared from metals and alloys of known composition, most commonly pure Au, Ag or, rarely, Cu, Pd or Pt.

To date no publication or invention claims the use of this phenomenon in the inverted approach, whereby a chemical compound or a mixture of such with known characteristics is utilized to analyze and characterize an alloy of coinage metals of unknown characteristics or other substrate with the results of analysis supplying information about the presence of such metals in the alloy and their relative proportions in the alloy.

This inverted approach is, in essence, the principal claim of the presented invention: instead of using known metals to establish the identity and properties of an unknown chemical compound, the disclosed invention uses a known chemical compound that is applied onto a surface of unknown properties, composition and identity. The known chemical compound is not chosen randomly but rather according to the set of criteria that form an algorithm allowing to choose the most appropriate and effective chemical compound. The said criteria are incorporated into this disclosure as an essential and necessary part of the invention and can be briefly described thus: the chemical compound is chosen so that it allows for the phenomenon of surface enhancement to occur and it is chosen so that different degrees and different types of surface enhancement are observed when the chemical compound is applied on different surfaces, preferably such different degrees and types are uniquely associated with specific components of the surface. The full set of criteria is found in the Detailed Description of the Drawings' section of this disclosure.

The practical need for a method and apparatus that allows confirming or denying presence and relative abundance of coinage metals in a sample of an alloy stems from the high economic value of said metals, especially gold.

One of the practical needs this invention meets is the need for a non-destructive, non-toxic, inexpensive assay of metallurgic composition of such alloys. It is commonly known that alloys of coinage metals enjoy widespread applications in the field of electronics, where coinage metals and their alloys are extensively used, as well as the field of banking, where gold and silver are used as investment instruments, as well as the field of jewelry, design and fashion. According to the World Gold Forum, 50% of all newly minted gold is used to manufacture jewelry, 40% is used as investment and the remaining 10% is used in industry.

In the field of jewelry, gold is commonly alloyed with other coinage metals and other metals, notably Zinc, Nickel and Palladium, forming gold alloys that possess desirable mechanical and chemical properties. In the United States, as well as throughout the world, specific standards have been codified in regard of alloys of gold and silver: only alloys of established content of gold and silver are admitted. The most common alloys of gold that are legally allowed to be marked as such in the USA are 10K, 14K and 18K, corresponding to 10, 14 and 18 parts of gold by weight in the total of 24 parts of metals of which the alloy is composed. This approximately corresponds to alloys with 41.(6), 58.5 and 75.0 weight percent of gold in the alloy. For silver, the most common alloys are Coin (90.0 weight percent of silver in the alloy), Sterling (92.5 weight percent of silver in the alloy) and Fine (99 weight percent of silver in the alloy).

The need to establish factual presence and weight ratios of gold and silver in the various alloys is well recognized in the field of jewelry manufacturing and trade as well as industrial manufacturing and trade.

Historically, several assays have been developed to measure the gold content of an alloy of coinage metals; however, all of these assays possess certain undesirable characteristics.

The most ancient of gold assays is the volumetric method, ascribed to the great scientist of antiquity, Archimedes. As the density of gold is exceptionally high, the Archimedes method allows, under certain circumstances, detection of the presence of gold; however, it does not allow for reliable and quantitative measurement of weight percent of gold in most common alloys.

Another ancient assay, very commonly used in modern practice, is the touchstone assay, in which the sample of an alloy is rubbed against a stone and the resulting fine film of the alloy is subjected to acid attack, commonly with mixtures of nitric and hydrochloric acids. The sensitivity of the touchstone assay is sufficient for determination of gold content with precision up to 0.5%; however, the assay is partially destructive, as a small but measurable amount of the alloy is lost, as well as labor-intensive, operator-dependent and utilizing highly corrosive and toxic acids.

The cupellation assay is yet more precise; however, it is fully destructive, as the sample is decomposed to produce pure gold, which is then weighted; it is also reliant on toxic compounds, such as lead oxide and metallic lead. The high temperatures utilized in the assay also pose a challenge. It is also not applicable to coatings, especially when the total expected amount of gold is less than a fraction of a gram, which is practically always the case.

Assays that are more modern include X-ray fluorescence, a highly precise assay that is hindered by the use of potentially dangerous penetrating radiation and the high cost of equipment. Another commonly used group of assays is the electrochemical assays that rely on the exceptionally high electrode potential of gold. The electrochemical assays also suffer from a number of shortcomings, such as the use of unstable and toxic compounds (oxidizers, such as hydrogen peroxide and related substances) as well as the issue of electrochemical transfer of atoms of gold and other metals from the measured sample to the reference electrode, which reduces sensitivity in a non-linear fashion.

An example of a non-destructive assay is the assay disclosed in the U.S. Pat. No. 8,673,126 issued to L. Radomyshelsky and B. Loginov, in which the electrochemical properties of the alloy are determined utilizing a corrosive and toxic electrolyte, which, as the inventors admit, must be contained and controlled.

Another example of a non-destructive assay is the assay disclosed in the U.S. Pat. No. 6,051,126 issued to L. V. Fegan, Jr. in which electrochemical properties of the alloy are determined with the use of an electrolyte that is described as corrosive.

Both of these assays may be used to characterize coatings; however, both pose a significant risk of destruction of the coating either by electrochemical solubilization or by corrosion.

The need for a non-destructive assay is met by the disclosed invention that utilizes non-toxic chemicals, does not involve the use of ionizing radiation, is sensitive, specific and reliable, can be performed at room temperature or other relatively harmless conditions and does not suffer from the risk of transfer of atoms of the analyzed sample to the reference electrodes or solubilization of the substantial portion of the analyzed sample.

### SUMMARY AND OBJECTS OF THE INVENTION

It is a primary object of the present invention to provide a method for detection of the presence and quantitative determination of the relative amounts of chemical elements including coinage metals (gold, silver and copper) as well as the platinum group metals (ruthenium, rhodium, palladium, osmium, iridium, and platinum) in materials inclusive of alloys of unknown or insufficiently known composition, including alloys that exist in the form of coatings, such as thin films. Additionally, other chemical elements may be detected, including detection in dielectric materials.

The disclosed invention utilizes the phenomenon known as ‘surface enhancement’, in which physical and chemical properties of a chemical reagent change upon application of such reagent onto the surface of a material. By using a reagent with known properties and detecting the change of its properties upon application on the surface of the tested object, it becomes possible to establish identity of the elements present in the tested object, as well as their relative abundance, or specific composition of the alloy or mixture of elements. The process of choosing of a reagent is explicated in this disclosure and may include the choice of specific patterns of change of physical and chemical properties attributed to surface enhancement, identity of atoms and functional groups of the reagent which may bind, coordinate or otherwise adhere to the surface of the tested material, specific distances between atoms and functional groups of the reagent that may correspond to the characteristics of the atomic lattice or crystalline structure of the tested material. The reagent may be chosen from already known chemical substances or specifically synthesized de novo.

One embodiment of the disclosed invention uses a physical process, known as surface enhanced Raman spectroscopy (SERS), in an apparatus that allows for detection and measurement of changes in the SERS spectrum of a known compound applied onto the surface of the tested alloy as well as comparison of the SERS spectrum of the known compound prior to application and after the application on the surface of the tested alloy, as well as qualitative computation of the composition of the alloy based on the collected data.

These and other objects and features of the present invention will become more fully apparent from the following description, or may be learned by the practice of the invention as set hereinafter.

### BRIEF DESCRIPTION OF THE DRAWINGS

In order to illustrate the manner in which the above-recited and other advantages and objects of the invention are obtained, a more particular description of the invention briefly described above will be rendered by reference to several embodiments thereof, which are illustrated in the appended drawings with the understanding that these drawings depict only typical embodiments of the invention and are not therefore to be considered limiting in scope. The invention will be described and explained with additional specific detail through the use of the accompanying drawings in which:

FIG. 1 is a schematic illustration of the apparatus that allows the detection and quantification of the surface enhanced Raman spectrum of a Reagent compound applied on the surface of the tested alloy;

FIG. 2 is a schematic illustration of the changes in the surface enhanced Raman spectrum of a Reagent compound when it is applied on surfaces of alloy that contains atoms of gold, silver, and copper compared to the Raman spectrum of the compound in its bulk, unapplied form;

FIG. 3 illustrates the principles of choosing the Reagent compound based on its constituent atoms and their ability to form coordinated complexes with atoms of the tested alloy as well as relative distances between atoms and functional groups of the Reagent compound, a set of principles essential to the disclosed invention.

### DESCRIPTION OF EMBODIMENTS

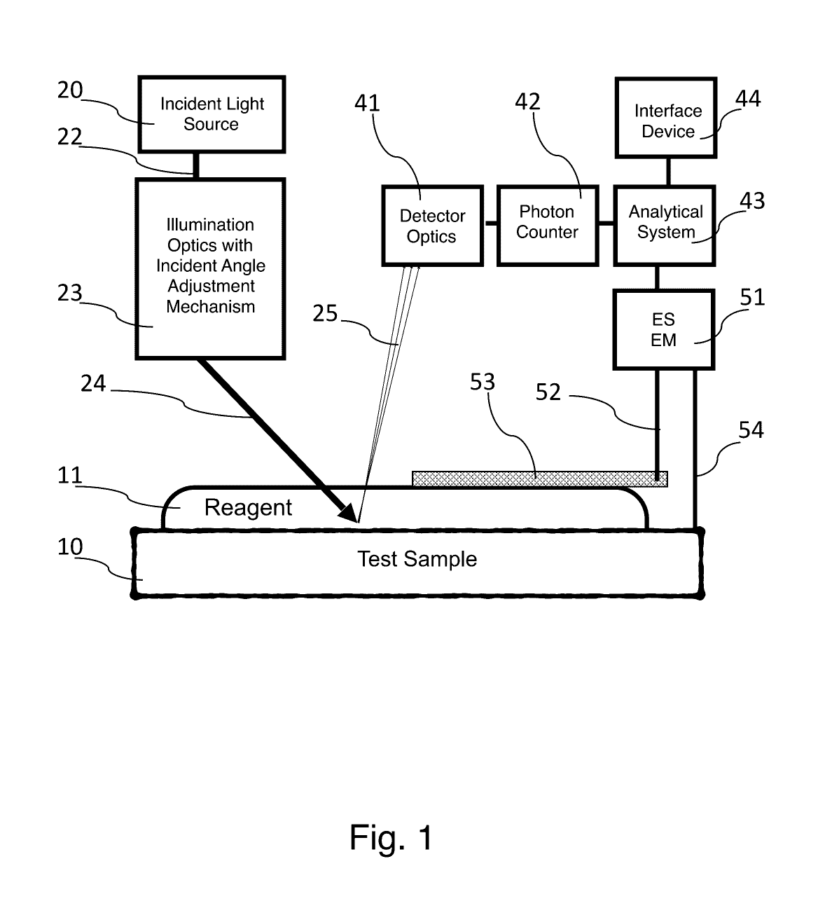

One embodiment of the disclosed invention is hereby explicated. FIG. 1 depicts an embodiment in which surface enhancement Raman scattering (SERS), a physical process, is utilized according to the disclosed method.

The disclosed apparatus is designed to test the presence of coinage metals (gold, silver, and copper) as well as platinum group metals in the Test Sample 10 by application of the Reagent compound 11 on the surface of the Test Sample 10. The disclosed apparatus consists of the Source of Incident Light 20, producing a Beam of Light 22 that is focused and modified by the Optical System 23 that allows illumination of the Reagent 11 with incident light and contains focusing assembly and mechanism allowing for adjustment of the angle at which the Incident Beam of Light 24 strikes the Reagent 11 that is adsorbed on the surface of the Test Sample 10. The Scattered Light 25 is captured by the Detector Optics 41, which includes a narrowband optical filter that rejects light of specific wavelengths as well as a spectroscopic setup that separates the scattered photons on the basis of their wavelength, such as by the use of optical slits or splitters, diffraction gratings, prisms or elements of non-linear optics. Scattered light captured by the Detector Optics 41 is directed to the Photon Counter 42 which measures the number of photons of specific wavelengths and transmits the counts to the Analytical System 43 comprised of computing hardware and software that establishes positions and intensities of characteristic peaks of the spectrum and transmits this information to the Interface Device 44 that relays the information to the human operator (not shown) or subsequent analytical hardware and software (not shown).

Additionally, an electrochemical assembly is implemented that is controlled by the Analytical System 43 that, if the option is implemented, applies electric current of specific characteristics (such as voltage, current, duration and shape of the current pulses and other parameters) to the Reagent 11 by controlling the Electric Source and current/potential measuring device (galvanometer) 51 that supplies the electric tension via Electric Conduit 54 connected to the Test Sample 10 and via the Electric Conduit 52 to the Counterelectrode 53 that is immersed in the Reagent 11 without being in direct contact with the Test Sample 10.

Optionally (not shown) the apparatus may contain conveniences not essential for the disclosed invention such as a sample holder to which the Test Sample 10 is affixed and various structural elements such as mechanical supports, mechanical stages facilitating movement of the sample, mechanical and optical barriers, liquid handling devices (such as droppers and wipers), other parts allowing for a steady interconnection and arrangement of the parts of the apparatus, as well as movement of the test sample 10, application of the specific amounts of the Reagent 11, prevention or vice versa, facilitation of evaporation of the Reagent 11, as well as constructive components that provide mechanical stability, rejection of stray and ambient light, reduce fatigue of the human operator, if present, and other functions that may be desirable.

The sequence of detection of the presence and quantification of chemical elements may include the following steps:

A. Test Sample 10 such as a sample of a metal alloy or an object manufactured from a metal alloy or object coated with a film of a metal alloy or a dielectric substance to be analyzed is cleaned from contaminants, both inorganic (including oxides and sulfides; especially, if presence of silver is suspected, from silver sulfide) and organic (including skin oils due to handling of the sample). This is readily accomplished by processes, known in the art, for example, cleaning of an object manufactured from an alloy of coinage metals is accomplished by immersion and agitation in an organic solvent bath, followed by a mild acid bath, followed by ultrasonic agitation in several baths of pure water with subsequent drying in a dust-free atmosphere or other cleaning procedures appropriate for the material being analyzed;

B. Test Sample 10 is placed inside the apparatus in a fashion that prevents the sample from being damaged and yet allows for operation of the apparatus;

C. A sufficient quantity of the Reagent 11 is applied on the cleaned surface of the test sample and may be subject to optional mechanical conditioning (such as spreading into a thin layer), physical conditioning (as described in steps D and E) or other conditioning (such as evaporation of the solvent if the Reagent compound is applied as a solution);

D. (OPTIONAL) An Electric Conduit 54 is attached to the Test Sample 10, while the Counterelectrode 53 is immersed in the Reagent 11, thus forming an electrochemical circuit;

E. (OPTIONAL) A reduction-oxidation cycle is run whereby the Test Sample 10 and the Reagent 11 are subject to electric potentials generated by the Electric Source 51 that variate polarity and voltage to facilitate electrochemical roughening of the surface of the Test Sample 10. Typical cycle, subject to modification, is +0.6V for several seconds, followed by −0.8V for several seconds. The details of one of the possible protocols of the reduction-oxidation cycle may be found in the publication by M. Fleischmann et al. (Citation 4) and are subject to variation depending on the specific Reagent compound. The cycle is controlled by the Analytical System 43 and may be repeated several times;

F. The spot of the Reagent 11 on the surface of the Test Sample 10 is illuminated with monochromatic Beam of Light 24 from the Source 20 as delivered by the Illumination Optics 23 at the specific incident angle which may remain constant during this step or may be implemented as varied or scanning during this step;

G. (SIMULTANEOUSLY with Step F). Scattered Light 25 emanating from the interface of the Reagent 11 with the surface of the Test Sample 10 is collected by the Detector Optics 41, with elastically (Rayleigh) scattered light of the same wavelength as the incident light rejected by the Detector Optics 41, while inelastically (Raman) scattered light is transmitted and subjected to spatial separation based on the wavelength by the same Detector Optics 41. Such spatial separation may be accomplished by the means traditional in the art of spectroscopy, including passing the transmitted light through a slit with subsequent separation on one or more diffraction gratings, with other, more exotic methods also possible;

H. (SIMULTANEOUSLY with Steps F and G). Transmitted and spatially separated photons of the inelastically (Raman) scattered light are captured by the Photon Counter 42, which measures the absolute counts of photons of specific wavelengths, creating a mathematical description of the spectrum of Scattered Light 25. The choice of specific wavelengths depends on the choice of the reagent compound. The photon counter may be implemented as a vacuum photomultiplier, a charge-coupled device or any combination thereof, as well as a complementary metal oxide semiconductor image sensors, including those that utilize stacked geometry as taught by Richard B. Merrill in U.S. Pat. No. 6,632,701 and related inventions;

I. The absolute counts of photons of specific wavelengths are analyzed by the Analytical System 43 comprised of computer hardware and software to produce the desired results of the analysis, preferably in the form of a table of weight percentages of specific coinage metals, platinum group metals or other identifiable components of the test sample and the results of analysis are relayed to the Interface Device 44, such as a computer monitor, printing device or any other desired recipient of information (not shown);

J. (OPTIONAL) In an optional protocol, the electrochemical circuit comprised of 51, 52, 53, and 54 is activated by a subroutine of the computer software of the Analytical System 43, resulting in development of electric potential in the system comprised of the Test Sample 10 (functioning as the working electrode)—Reagent 11—Counterelectrode 53. In this circuit the surface of the Test Sample 10 may be supplied with electric potential ranging from −0.1V to −1.4V, with the numerical value being preselected based on the specific properties of the Reagent 11. Steps F, G, H, and I are then repeated under the conditions of applied electric potential.

K. (OPTIONAL) Steps J, F, G, H, and I may also be repeated with a different numerical value of the applied electric potential.

In the process of preliminary research for the disclosed invention, a total of 206 chemical compounds were evaluated for suitability to serve as the Reagent 11. The following is a brief summary of the results for two known chemical compounds and one specifically synthesized compounds that illustrates the feasibility of the disclosed invention and its first specific embodiment utilizing surface enhancement of Raman scattering.

The first successfully tested compound was the sodium salt of 2-sulfanylethanesulfonic acid, also known as MESNA, represented by the structural formula of HS—(CH₂)₂—SO₃Na, a relatively non-toxic substance that meets the criteria, formulated for the Reagent (criteria i, ii, and iii as explicated below) as a 5% solution in unbuffered deionized water. The sole Anchor Atom expected to coordinate with the surface of the test sample is the atom of sulfur of the —SH (sulfohydryl) group, which has great affinity for atoms of silver, moderate affinity for atoms of copper and weak affinity for atoms of gold.

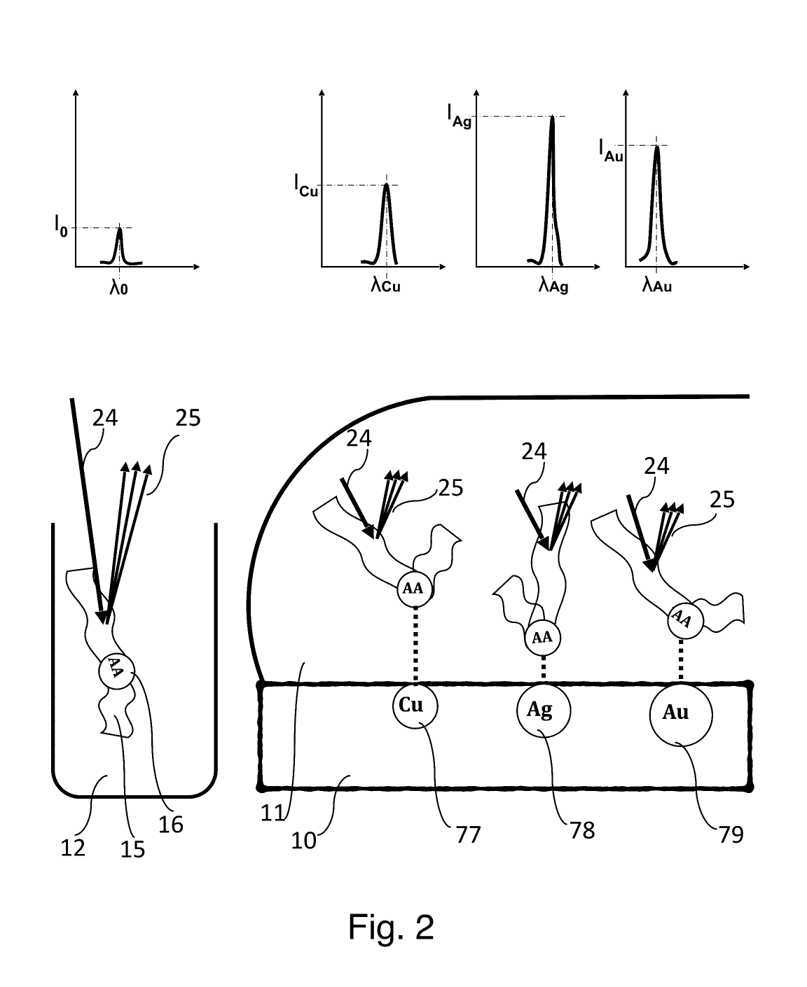

The representation of the results of the experiment is depicted in FIG. 2 which presents the schematics of two experiments that were performed utilizing the disclosed methods and apparatus. In the first part of the experiment, the Reagent is present in its Bulk form 12. The Reagent molecule 15 is depicted as possessing one Anchor Atom 16 (also denoted as AA) that is capable of coordination with atoms and molecules that may be present in the Test Sample. Illumination of the Reagent in its bulk form 12 in the absence of a Test Sample with monochromatic incident Beam of Light 24 and collection of the Raman Scattered Light 25 produced data that was plotted as Intensity (number of scattered photons) vs. wavelength (plot directly above). This is commonly done to acquire the Raman spectra of various compounds. For illustration purposes, only one peak is depicted, characterized by intensity I₀ and central wavelength λ₀.

In the next step, the Reagent 11 is applied to the Test Sample 10, containing atoms of copper 77, atoms of silver 78, and atoms of gold 79. The Anchor Atom 15 (denoted as AA) of the reagent compound 11 enters into a coordinated state with atoms of the surface of the sample. With this coordination of the Anchor Atom 16 with the atoms of the test sample, the molecule of the Reagent 15 is geometrically distoreted and two distinct types of surface enhancement quantum mechanical phenomena are observed due to surface enhancement of the Raman scattering. The first phenomenon is the amplification of the intensity of the peak thus, indicated as I_{Cu}, I_{Ag}, and I_{Au}; while the second phenomenon is the shifting of the central frequency of the peak thus, indicated as λ_{Cu}, λ_{Ag}, and λ_{Au}. With mathematical analysis, the shifting of the frequency allows for identification of the specific metal's presence, while the relative intensities of the peaks allow for determination of the content of specific metals in the alloy.

In the first experiment, the Raman spectrum of the sodium salt of 2-mercaptoethanesulfonic acid (MESNA) was measured as a bulk solution in water, and subsequently was measured when said solution was applied on the surfaces of samples of pure gold, pure silver and pure copper, and subjected to illumination with a 532 nm monochromatic laser light. The following peaks were recorded: in solution—551.42 nm (peak A) and 553.86 nm (peak B); on pure gold—550.84 nm (shifted peak A) and 552.82 nm (shifted peak B); on pure silver—550.69 nm (shifted peak A) and 552.76 nm (shifted peak B); on pure copper—550.39 nm (shifted peak A) and 552.27 nm (shifted peak B).

Such peaks can also be described using the wavenumber convention popularly used in scientific publications in the field of Raman spectroscopy as 662-A and 742-B (in solution); 643-A and 718-B (on Au); 638-A and 706-B (on Ag); 628-A and 690-B (on Cu). The relative intensity of the peaks was no less than 10³ times stronger compared to the peaks measured in solution. The highly sensitive spectroscopic setup (41, 42, 43, 44) used in the experiment allowed for separation of peaks differing in 6 wavenumbers (approximately 0.17 nm of wavelength).

The results of the experiment allowed for the proof of principle, as distinct peaks corresponded to different coinage metals. The rationale behind the distinction is that due to coordination of the molecule of the reagent with the atoms of metals, characteristic peaks A and B shift their position due to energy transfer between the molecule and the coordinated atom, with the amounts of such transferred energy depending on the relative strength of the coordination bond or other quantum mechanical effects. In this experiment, peak A shifted from its expected position (such as measured in solution with no metals present) by 19 wavenumbers when the Reagent was on the surface of metallic gold, by 24 wavenumbers for silver, and 34 wavenumbers for copper; while peak B shifted by 24 wavenumbers for gold, 36 wavenumbers for silver, and 52 wavenumbers for copper.

These results alone allow for identification of a specific coinage metal by the action of the disclosed apparatus in accordance with the disclosed method.

The second tested compound was uracil, a heterocyclic chemical of minimal toxicity and molecular formula C₄H₄N₂O₂. In this compound, the Anchor Atom is one of the atoms of nitrogen, with both atoms of oxygen also co-coordinating on the surface of the metallic alloy.

The schematics of the experiment correspond to the schematics explicated above for the experiment involving MESNA with the exception that Raman spectrum was not obtained from the solution of uracil in water, as this spectrum is readily available from reference sources.

Upon application of uracil onto the surfaces of samples of pure gold, pure silver and pure copper, and illumination with a 532 nm monochromatic light, the following peaks were recorded: on pure gold—574.38 nm (peak A) and 580.89 nm (peak B); on pure silver—574.88 nm (peak A) and 580.42 nm (peak B); on pure copper—572.84 nm (peak A) and 581.84 nm (peak B). Such peaks can also be described using the wavenumber convention as 1387-A (on Au) and 1582-B (on Au), 1402-A (on Ag) and 1568-B (on Ag), and 1350-A (on Cu) and 1590-B (on Cu) correspondingly. The separation of peaks is sufficient for identification of metals, thus the second experiment was performed, in which a solution of uracil was applied on an alloy of 58.5 weight percent of gold, 20.75 weight percent of silver and 20.75 weight percent of copper (an alloy that qualifies as 14K gold under current regulations). The alloy was prepared in bulk by slow melting under argon with ultrasonic agitation of the liquidus to assure the uniformity of the alloy and reduction of loss, especially of copper, due to oxidation and sputtering, with the total weight of the prepared alloy sample of 121.4 grams (approximately 4 troy ounces). The weight percept composition of this alloy of 58.5% gold, 20.75% silver and 20.75% copper corresponds to the atomic percentages of 36.40% gold, 23.58% silver and 40.02% copper.

The relative intensity of the peaks obtained from uracil as applied on the surface of such 14K gold alloy was compared with the relative intensity of the peaks, obtained from uracil when applied on pure constituent metals. Specifically, the peak at 574.88 nm (wavenumber 1402), which is peak A as observed on silver, was registered in one measurement series at 21050 counts on pure silver and 5003 counts on the alloy, indicating the calculated atomic percentage of silver in the alloy at 23.77%, in agreement with the expected atomic concentration of 23.58% which corresponds to weight concentration of 20.75%.

The results of these experiments allowed formulating the following list of criteria for the properties and specifications of the Reagent allowing either to choose the Reagent from a list of candidate compounds or to direct the synthesis of a previously unknown chemical compound.

The relevant specifications include the parameters of the Reagent as a single compound or a mixture of compounds. These parameters form a set and application of these parameters forms an algorithm that is an essential and indelible part of the disclosed invention. All or some of these parameters may be applied in determining the choice of the Reagent as a single compound or a mixture of molecular species to be utilized in the operation of the disclosed apparatus. These parameters may also be utilized in designing a novel (previously unknown) compound or compounds to serve as the Reagent in the operation of the disclosed apparatus. Such parameters include:

i. The presence of surface enhancement or geometric shape changes (such as overall shape and relative positions of atoms and functional groups of the compound or compounds) when such Reagent is applied on the surface of a tested solid object and a relevant physical or chemical process takes place;

ii. The presence of different degrees or types of surface enhancement or changes of the geometric shape of the molecule or molecules of the Reagent when the Reagent is applied onto surfaces of different substances including different metals of the coinage group (gold (Au), silver (Ag) and copper (Cu)), platinum group (ruthenium (Ru), rhodium (Rh), palladium (Pd), osmium (Os), iridium (Ir) and platinum (Pt)) or other chemical elements and their compounds; preferably, the degree and type of surface enhancement and/or change of the geometric shape of the molecule of the Reagent should be unique and specific for each of the aforementioned metals or other chemical elements and their compounds; also, preferably, such unique and specific degrees, types and changes should be discernable by observation, measurement of induction of secondary processes inclusive of physical changes (for example, phase, conductivity, reflectance and other discernable physical characteristics) and chemical changes (for example, initiation or termination of a chemical reaction, electrochemical potential change, luminescence and other manifestations);

iii. The presence of one or several anchor atoms in the molecular structure of the Reagent which are defined as atoms present in the molecular structure of the Reagent that are capable of coordination with the atoms of the tested solid object thus altering the structure and properties of the Reagent, whereby said anchor atoms are chosen from atoms of elements of the Periodic Table with preferentially chosen elements belonging to the Group 1 (Hydrogen and alkali metals, such as Lithium, Sodium, Potassium, Rubidium and Cesium); Group 2 (2A) (also known as alkaline earth metals, such as Beryllium, Magnesium, Calcium, Strontium, and Barium); Group 14 (4A) (also known as tetrels or tetragens, such as Carbon, Silicon, Germanium, Tin and Lead); Group 15 (5A) (also known as pnictogens, such as Nitrogen, Phosphorus, Arsenic, Antimony and Bismuth); Group 16 (6A) (also known as chalcogens, such as Oxygen, Sulfur, Selenium and Tellurium); Group 17 (7A) (also known as halogens, such as Fluorine, Chlorine, Bromine, and Iodine) of the Periodic Table or other chemical elements;

iv. The presence of a reliable and repeatable changes in surface enhancement or changes in the geometric shape of the molecules of the Reagent observed when the Reagent is applied on the surfaces of the tested object and a relevant physical or chemical process takes place producing readily analyzable data in predictable and measurable relation to the identity of the atomic and molecular components of the tested object;

v. The technical ease of the use of solutions of the Reagent that are formulated in inorganic and organic solvents presenting with different physical (such as internal entropy, optical opacity, drying time, and electric conductivity) and chemical (such as pH, stability in atmosphere, stability to photic degradation, formation of stable monolayers or bilayers, and oxidative capacity) properties;

vi. The possibility of incorporation of stable isotopes of the anchor atoms as present in the molecular structure of the Reagent, such as deuterium in place of protium (the common isotopes of Hydrogen), isotopes of Carbon, such as C¹³; Oxygen, such as O¹⁸; Nitrogen, such as N¹⁵; Sulfur, such as S³⁴, and others with the goal of achieving discernible and reliable difference in data pertaining to surface enhancement or geometric shape change of the molecules of the Reagent obtained when the Reagent is applied on the surface of the tested solid object compared with the data obtained from a sample of the Reagent that is not applied on a surface of a test object or applied on a different object, such on the surface of a control object or etalon, including etalons manufactured from pure coinage and platinum group metals;

vii. Specific and determinable geometric dimensions of its molecule or molecular species (if a mixture of such is utilized) so that the molecule or molecular species present with geometric dimensions commensurable with the geometric dimensions of the elements of the fine structure of the tested material, especially the lattice parameters and diagonal measurements of lattices that are reasonably anticipated to be present in the tested material should it contain chemical elements of interest, one example being the lattice of the crystalline structure of pure gold (Au) composed of cubical elements with the lattice parameter of 0.40782 nanometers as measured (Citation 5), in which case the molecule or molecular species of the Reagent may be most desirable if the geometric dimensions of these molecules or molecular species can be described by the formula D 0.40782a+0.57674b+0.70636c (nanometers), whereas a, b, and c are coefficients applied to the lattice parameter, the face diagonal of the lattice and the space diagonal of the lattice of the pure gold sample;

viii. Optionally, the parameters for choosing or synthesizing the Reagent may include the presence of specific chemical structural groups in its molecules, such as, in one example, chains of atoms forming conjugated molecular bonds, such as, in one implementation, chains of carbon forming conjugated p-orbitals with delocalized electrons, serving to further enhance such physical processes as inelastic scattering of light (Raman scattering);

ix. Optionally, nanoparticles as contrasted to solutions or bulk forms of chemical elements and molecules may be utilized as the Reagent, such as nanoparticles of gold, silver, carbon, silicon and their compounds, with the said nanoparticles used in their native form or in their coated form, in which the coating applied onto the said nanoparticles may be one or more of the chemical compounds that meet the criterions formulated above and especially criterions i, ii, iii, and iv that are essential for this application of nanotechnology.

Based on these criteria, the synthesis of a previously unknown compound was attempted with the goal of increased sensitivity and specificity of measurements. This compound, labeled as ‘sulfurated xanthophyll’, was prepared specifically for the experiments and was chosen due to the fact that it conforms to the aforementioned criteria, especially i, ii, iii, iv, v and viii. This compound has the bulk chemical formula of C₃₆H₄₄S₂, and is notable for the presence of two anchor atoms, both being sulfur, as well as two aromatic rings connected by a chain of conjugated (double-single) carbon-carbon bonds. The compound was synthesized using a modification of the method described for the synthesis of 3′-thiolutein (Citation 6). The choice of the compound was dictated by the fact that the presence of two atoms of sulfur will allow coordination with not one, but two atoms of silver, for which sulfur has the strongest affinity. The atoms of sulfur are located at the ends of the molecule, and the length of the carbon chain that connects them is more than the diagonal of the smallest element of the crystal lattice of the tested alloys, thus corresponding to the criterion vii.

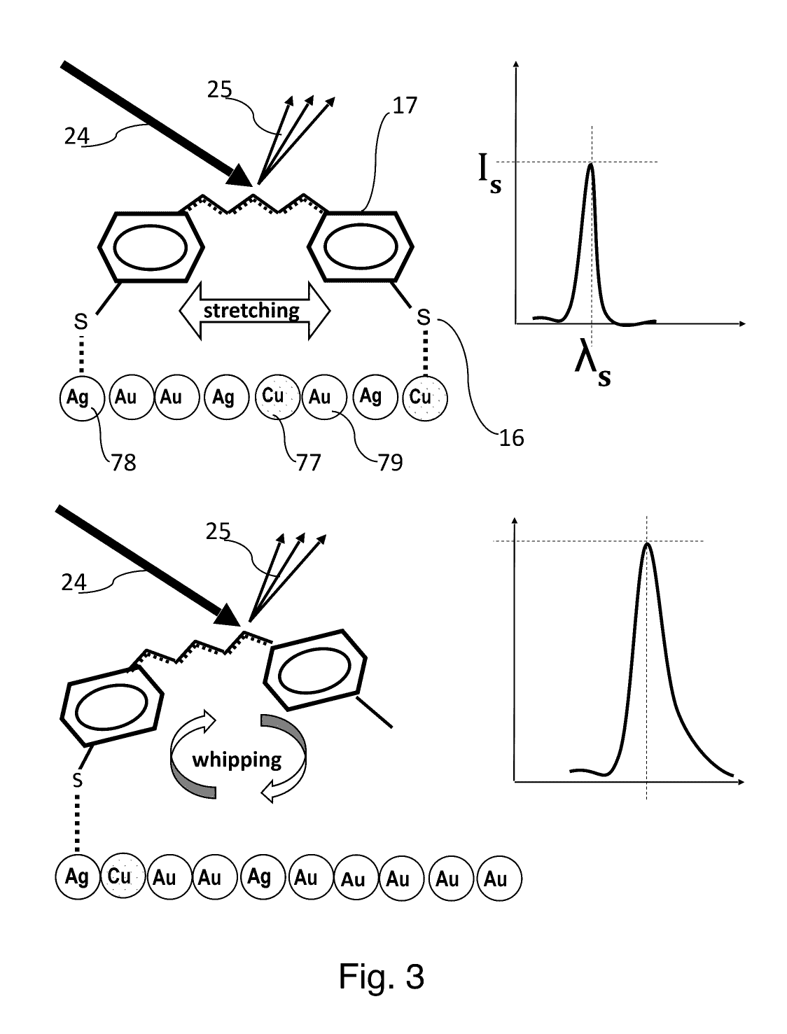

This consideration allowed for a larger variation of observed Raman peaks, as illustrated in FIG. 3.

In this schematic illustration, two molecules of the sulfurated xanthophyll 17 are coordinated by the atoms of sulfur 16 with the atoms of copper 77, atoms of silver 78 and atoms of gold 79, as present on the surface of an alloy of these metals. Given the strong affinity of sulfur for silver and weak affinity for gold, the molecule depicted in the top drawing is anchored to the surface in two spots, while the molecule depicted in the bottom drawing is anchored in one spot only. The resulting vibrations of the molecules differ, with the molecule that is anchored twice being more likely to experience one type of vibrations, termed “stretching”; while the molecule anchored only on one side is more likely to experience “whipping” vibrations. Note that terminology, as well as the schematic depictions in FIG. 3 inclusive of the localization of peaks, are for illustration purposes only; as anchor atoms other than sulfur are not shown, the measurements of the molecule and the exact number of conjugated bonds are depicted in a simplified fashion sufficient to illustrate the principles as disclosed hereby.

As a result of this carefully considered topology of the molecule of sulfurated xanthophyll, which has been synthesized specifically for these experiments, the following was found:

The most prominent peak of the Raman spectrum of sulfurated xanthophyll in solution was observed at wavenumber 1520 and termed as Peak A with a remarkably high strength of the signal (approaching 200,000 counts) due to the internal resonance along the conjugated carbon-carbon bonds. On application on pure gold, the peak shifted to 1530 and increased its strength by the factor of 7.9. On application on pure silver, the peak shifted to 1575 and increased its strength by the factor of ˜20. On application on pure copper, the peak shifted to 1555 and increased its strength by the factor of 4.8. Thus, the peaks were separated by 25 wavenumbers (Au and Cu) and 20 wavenumbers (Cu and Ag), which is sufficient to detect even with an inexpensive Raman spectrometer.

Furthermore, application of this compound on the previously described alloy of 14K gold allowed the direct measurement of content of gold, silver and copper based on the relative intensity of the peaks.

In addition, when the electrostatic potentials increasing from −0.2V to −1.4V in a step-wise fashion were applied on the sample (as described in Steps J and K), the intensity of the peak increased significantly allowing the sample at a certain point to be identified as containing between 58.3 weight % and 58.7 weight % of gold, 20.7 weight % to 20.8 weight % of silver and 21.0 weight % to 22.2 weight % of copper, a result that exceeds the results obtained from the same sample by the touchstone assay and is on par with the results obtained by X-ray chromatography.

With the same reagent dissolved in a non-polar solvent, additional experiments were performed, this time attempting to discern solid samples of alloys of gold from electrochemically deposited films of gold alloys on a foundation of solid copper, an important practical goal. The test and control samples used in the experiment were manufactured from a sheet of pure copper (test sample) or pure gold (control sample) that was coated with an alloy of gold and nickel by electroplating it in a commercially available electrolyte (Midas 14K Yellow Bright Gold Plating Solution, Acid Based; Rio Grande, Inc. of Albuquerque, N. Mex., USA). This electrolyte contains ions of gold and nickel complexed to cyanide ions, as well as some non-metallic additives. The deposited layer of gold alloy is expected to match the color of the 14K solid sample and was, indeed, found to be a good match. As there is no copper in the electrolyte solution and care was taken not to use any copper-containing parts in the setup of a electroplating apparatus whereas all conductive elements were either platinum or platinized titanium, should the presence of copper be detected by the experiment, it would suggest the ability to “see through” the gold plate and detect the underlying material. The thickness of the gold alloy plate was estimated according to the manufacturer's instructive pamphlet as being between 100 and 150 nm. Under the US Federal Trade Commission's rules, such gold plated item can be marked as “Gold Wash/Gold Flashed” (thickness of the layer of the gold alloy is lower than 175 nm). Same procedure was followed to produce a control sample on a sheet of pure gold, with the results noted as looking very similar both to the organoleptic examination (sight and touch) and detailed visual examination under 10× magnification.

The gold plated sheets were washed several times in acetone, followed by a wash in 5% solution of citric acid and several washes in pure water. Upon drying the surface was examined under 10× magnification and was found to be uniform without visible defects or pores. The experiment proceeded under the same conditions as described above with the exception of the variation of the angle at which the incident light 24 strikes the reagent 11, with the extent of variation being between −170 and −110 degrees. The purpose of such variation was the generation of an evanescent wave in the interface between the test sample 10 and the Reagent compound 11. It is expected that such angle is to differ depending on the specific chemical compound that is used as the Reagent, thus the technical embodiment of the apparatus, as described above, must allow for variable and controllable change of the incident angle. In one of the series of measurements, a wide peak with the maximum reasonably corresponding to that of copper was, indeed, detected at 1555 wavenumbers and was comparably strong (30,000 photon counts) compared to the peak of gold at 1530 wavenumbers and intensity of over 200,000 photon counts. Notably, the intensity of the peak at 1530 wavenumbers measured on the test sample of gold-plated copper was found to be lower compared to the control sample of the same coating applied on pure gold. The results of the experiment indicate that detection of the underlying material is possible for at least some types of gold plating without the need to remove such plating and that an evanescent wave may be generated under the described conditions.

It is understood that a large number of other compounds may present with desirable properties as to be utilized as Reagent 11 in the operation of the disclosed apparatus. Compounds tested include perylene derivatives known for the propensity to form self-assembling structures on gold and silver substrates, especially the perylene tetracarboxylic acid dianhydride (PTCDA) and its metal salts; molecules with complex three-dimensional geometry with examples being the derivatives of adamantane, nucleic acids, proteins, heme and other commonly encountered compounds. Additionally, numerous species of alkanethiols present with a promise due to the ability to form self-assembled layers on the surface of metals.

Potential refinements of the disclosed apparatus may include the use of adaptive optics, use of the alternatives to optical slits such as image slicers (Citation 7); use of different sectional geometries of the incident beam 24 such as the variations of the Gauss and Laguerre-Gauss beam profiles (Citation 8); application of heat to the reagent-sample interface; many-photon schemes, in which photons of two or more different wavelengths are utilized to excite the reagent-sample interface; the extension of the principle to testing of dielectric materials; use of variable wavelengths in the incident beam 24 allowing for better quality and strength of the scattered light 25 and other refinements that do not alter the main principle of the disclosed invention.

A distinct implementation of the disclosed method may utilize a different type of surface enhancement, namely, enhancement of chemiluminescence or electrochemiluminescence instead of enhancement of Raman scattering. In that case, the use of different chemical compounds is warranted, such as luminol and hydrogen peroxide; luciferin, luciferase and adenosine triphosphate; aequorin in the presence of calcium ions and oxygen; coordinated compounds of ruthenium, such as tris(bipyridine)ruthenium(II) cation in the presence of an applied electric potential or other compounds of desirable properties. The disclosed criteria i, ii, iv, vi, vi, and vii apply to the algorithm of selection of suitable chemical compounds.

Among technical details that may contribute to a more efficient and less costly implementation of the disclosed invention are the use of an etalon optical filter (also known as Fabry-Pérot etalon) tuned to reject most incidental light with the exception for the relevant wavelengths; such etalon optical filter will need to be paired with a specific Reagent.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes, which come within the meaning and range of equivalency of the claims, are to be embraced within their scope.

Although preferred embodiments of the invention have been described herein in detail, it will be understood by those skilled in the art that variations may be made thereto without departing from the spirit of the invention or the scope of the appended claims.

What is claimed and desired to be secured by United States Letters Patent is:

1. A method of analysis of materials utilizing the phenomenon of surface enhancement and geometric shape change of molecules coordinated and otherwise present on the surface of the tested material in a modality whereas a well-characterized reagent compound selected or synthesized for the purpose according to disclosed criteria explicated in claims 3 and 4 is applied onto the surface of a metal, metal alloy or a dielectric material and is subjected to a physical or chemical process that is expected to be affected by the phenomenon of surface enhancement with data related to the physical or chemical process collected and analyzed in comparison to previously known properties of the reagent compound thus revealing the identity of the metal, identity of the components of the metal alloy or chemical elements present in the dielectric as well as relative abundances of the identified components of the alloy as well as the composition and lattice arrangements of the dielectric material.

2. An apparatus utilizing the principle of the claim 1 that uses surface enhanced Raman scattering (also termed surface enhanced Raman spectroscopy) as the physical process expected to be affected by the phenomenon of surface enhancement and geometric shape change of the molecules coordinated and otherwise present on the surface of the tested material.

3. A criterion for selection or synthesis of the reagent compound according to claim 1, which include the presence of surface enhancement or geometric changes (such as overall shape and relative positions of various components of the compound or compounds) when such Reagent is applied on the surface of a tested object and a relevant physical or chemical process takes place.

4. A criterion for selection or synthesis of the reagent compound according to claim 1, which include the presence of a different degree of surface enhancement or change of the geometric shape of the molecule observed when the Reagent is applied on the surfaces of the tested object and a relevant physical or chemical process takes place and produces analyzable data with the aforementioned different degree or type of surface enhancement or change of the geometric shape of the molecule reliably and uniquely dependent on the identity and composition of the tested object such as the presence of specific chemical elements, especially coinage metals and platinum group metals, chemical functional groups, size and shape of crystal lattices or other structural elements, as well as electrochemical and quantum mechanical characteristics of the material being tested.
